(19) United States
(12) Patent Application Publication
(10) Pub. No.: US 2009/0318712 A1
(43) Pub. Date: Dec. 24, 2009

(54) CATALYST SYSTEM AND METHOD FOR PRODUCING CARBOXYLIC ACIDS AND/OR CARBOXYLIC ACID ANHYDRIDES

(75) Inventors: Hagen Wilmer, Ludwigshafen (DE); Cornelia Dobner, Ludwigshafen (DE); Tina Einfeld, Mannheim (DE); Sebastian Storck, Mannheim (DE); Jürgen Zühlke, Speyer (DE); Frank Rosowski, Mannheim (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/305,698

(22) PCT Filed: Jun. 6, 2007
(86) PCT No.: PCT/EP2007/055583

§ 371 Date: Dec. 19, 2008

(30) Foreign Application Priority Data
Jun. 20, 2006 (EP) 06115699.8

(51) Int. Cl. C07D 307/60; B01J 23/22; B01J 27/198; B01J 8/02; C07C 51/00

(52) U.S. Cl. 549/231; 422/218; 562/400

(57) ABSTRACT

Catalyst systems for preparing carboxylic acids and/or anhydrides, the catalyst system comprising a reaction zone and a layered catalyst, the reaction zone comprises a gas inlet region and a gas outlet region, the layered catalyst comprises an active composition and one or more middle layers, one or more first layers disposed on a side of the one or more middle layers toward the gas inlet region, and one or more second layers on a side of the one or more middle layers toward the gas outlet region, wherein the active composition content of one or more of the middle catalyst layers, based on total mass of the layered catalyst, is lower than the active composition content of the one or more first catalyst layers and is lower than one or more second catalyst layers; and processes for gas phase oxidation employing a layered catalyst of the present invention.

The present invention relates to a catalyst system for preparing carboxylic acids and/or carboxylic anhydrides which has at least three catalyst layers arranged one on top of the other in the reaction tube, with the proviso that the active composition content, based on the total mass of the catalyst, of one or more of the middle catalyst layer(s) is lower than the active composition content of one or more of the upper catalyst layer(s) toward the gas inlet side and is lower than one or more of the lower catalyst layer(s) toward the gas outlet side. The invention further relates to a process for gas phase oxidation in which a gaseous stream which comprises a hydrocarbon and molecular oxygen is passed through several catalyst layers, the active composition content, based on the total mass of the catalyst, of one or more of the middle catalyst layer(s) being lower than the active composition content of one or more of the upper catalyst layer(s) toward the gas inlet side and being lower than one or more of the lower catalyst layer(s) toward the gas outlet side.

A multitude of carboxylic acids and/or carboxylic anhydrides is prepared industrially by the catalytic gas phase oxidation of aromatic hydrocarbons such as benzene, the xylenes, naphthalene, toluene or durene in fixed bed reactors. In this way, it is possible to obtain, for example, benzoic acid, maleic anhydride, phthalic anhydride, isophthalic acid, terephthalic acid or pyromellithic anhydride. In general, a mixture of an oxygenous gas and the starting material to be oxidized is passed through tubes in which a bed of a catalyst is disposed. For temperature regulation, the tubes are surrounded by a heat carrier medium, for example a salt melt.

Even though the excess heat of reaction is removed by the heat carrier medium, local temperature maxima (hotspots) can be formed in the catalyst bed, in which there is a higher temperature than in the remaining part of the catalyst bed, or in the remaining part of the catalyst layer. These hotspots lead to side reactions, such as the total combustion of the starting material, or to the formation of undesired by-products which can be removed from the reaction product only at great cost and inconvenience, if at all. Moreover, the catalyst can be damaged irreversibly from a certain hotspot temperature.

To attenuate these hotspots, various measures have been taken. In particular, as described in DE 40 13 051 A1, there has been a transition to arranging catalysts of different activity layer by layer in the catalyst bed, the less active catalyst generally being disposed toward the gas inlet and the more active catalyst toward the gas outlet. In the case of a loading of 60 g/m³ (STP), a yield of 78 mol %, i.e. 108.8 m/m %, is achieved in a 2-layer catalyst system. The content of phthalide and residual o-xylene is not specified.

DE 198 23 262 A1 describes a process for preparing phthalic anhydride with at least three coated catalysts arranged in layers one on top of the other, the catalyst activity rising from layer to layer from the gas inlet side to the gas outlet side. At a loading of 85 g/m³ (STP), a yield of 113 m/m % in a 3-layer catalyst system is achieved. The content of phthalide is from 0.15 to 0.25 mol % in the crude PA, i.e. from 0.13 to 0.22% by weight in the reactor outlet gas. The content of residual o-xylene is not specified.

EP-A 1 063 222 describes a process for preparing phthalic anhydride which is performed in one or more fixed bed reactors.

The catalyst beds in the reactors have three or more than three individual catalyst layers in succession in the reactor. After passing through the first catalyst layer under the reaction conditions, from 30 to 70% by weight of the o-xylene, naphthalene or of the mixture of the two used has been converted. After the second layer, 70% by weight or more has been converted. At a loading of 100 μm³ (STP), a yield of >114 m/m % is achieved in a 3-layer catalyst system. The content of phthalide is 0.07 mol %, i.e. 0.06% by weight. The content of residual o-xylene is not specified.

EP-A 1 063 222 further summarizes that the activity rises as a result of the following measures or combinations thereof:

(1) as a result of constant rise in the phosphorus content,
(2) as a result of constant rise in the active composition content,
(3) as a result of constant decrease in the alkali content,
(4) as a result of constant decrease in the empty space between the individual catalysts,
(5) as a result of constant decrease in the content of inert substances or
(6) as a result of constant increase in the temperature from the upper layer (gas inlet) to the lower layer (gas outlet).

In principle, all catalysts lose activity with increasing lifetime as a result of aging processes. This predominantly affects the main reaction zone, since the highest thermal stress takes place there. In the course of this, the main reaction zone migrates over the course of the catalyst lifetime ever deeper into the catalyst bed. The result of this is that intermediates and by-products can no longer be fully converted, since the main reaction zone is now also within catalyst zones which are less selective and have enhanced activity. The product quality of the phthalic anhydride obtained thus worsens increasingly. It is possible to counteract the decline in the conversion and hence the worsening of the product quality by increasing the reaction temperature, for example by means of increasing the salt bath temperature. However, this temperature increase is associated with a decline in the yield of phthalic anhydride.

Moreover, the higher the loading of the air with the hydrocarbon to be oxidized, the greater the presence of intermediates and by-products, since a high loading enhances the migration of the main reaction zone deeper into the catalyst bed. For economically viable preparation, however, high loadings of from 80 to 120 g/m³ (STP) are desired.

The by-products which increase in the course of aging, especially in connection with a high loading, comprise not only phthalide (PHD) but also unconverted o-xylene.

EP-A 1 636 162 achieves a very small by-product spectrum, in particular very low values of anthraquinonedicarboxylic acid, by virtue of only the last catalyst layer comprising phosphorus and at least 10% by weight of vanadium (calculated as V₂O₅), based on the active composition of the catalyst, being present in the last layer, and the ratio of vanadium (calculated as V₂O₅) to phosphorus having a value of greater than 35. At a loading of 100 g/m³ (STP), in a 4-layer catalyst system, a yield of 113.5% is achieved with a residual o-xylene content of 0.003% by weight and a phthalide content of 0.02% by weight.

WO 2005/115616 describes a process for preparing phthalic anhydride in a fixed bed reactor having three or more catalyst layers with activity increasing in flow direction. It is disclosed that a small by-product spectrum is achieved when the content of the active compositions and hence the layer thicknesses of the catalysts decrease in flow direction. In the examples, at a loading of 60 g/m³ (STP), a yield of 113.7% is achieved in a 3-layer catalyst system. The phthalide content is <500 ppm, which corresponds to a value of 0.5% by weight. The content of residual o-xylene is not specified.

With regard to the general by-product formation at high loading and good yield, there is still a need for optimization. A reduction in these by-products additionally facilitates the workup of the crude phthalic anhydride.

It was therefore an object of the invention to provide a catalyst system and a process for preparing phthalic anhydride which, in spite of high loading, affords phthalic anhydride with improved product quality at the same or improved yield.

The object is achieved by a catalyst system for preparing carboxylic acids and/or carboxylic anhydrides which has at least three catalyst layers arranged one on top of the other in the reaction tube, with the proviso that the active composition content, based on the total mass of the catalyst, of one or more of the middle catalyst layer(s) is lower than the active composition content of one or more of the upper catalyst layer(s) toward the gas inlet side and is lower than one or more of the lower catalyst layer(s) toward the gas outlet side.

The active composition content of the middle layer(s) is advantageously from 0.1 to 5% by weight (absolute), preferably from 0.1 to 2.5% by weight, in particular from 0.3 to 1% by weight lower than the active composition content of the upper catalyst layer(s) toward the gas inlet side.

The active composition content of the middle layer(s) is advantageously from 0.1 to 5% by weight (absolute), preferably from 0.1 to 2.5% by weight, in particular from 0.3 to 1% by weight, lower than the active composition content of the upper catalyst layer(s) toward the gas outlet side.

The active composition content of the upper catalyst layer(s) toward the gas inlet side is advantageously from 5 to 15% by weight, preferably from 6 to 13% by weight, in particular from 7.5 to 10.5% by weight, based on the total mass of the catalyst.

The active composition content of the middle catalyst layer(s) is advantageously from 5 to 15% by weight, preferably from 6 to 13% by weight, in particular from 7 to 10.5% by weight, based on the total mass of the catalyst.

The active composition content of the lower catalyst layer(s) toward the gas inlet side is advantageously from 5 to 15% by weight, preferably from 6 to 13% by weight, in particular from 7.5 to 11% by weight, based on the total mass of the catalyst.

The BET surface area of the catalytically active components of the catalyst is advantageously in the range from 5 to 50 m²/g, preferably from 5 to 40 m/g, in particular from 9 to 35 m²/g.

The activity of the catalyst layers advantageously increases from the gas inlet side to the gas outlet side. If appropriate, catalysts inserted upstream or intermediately and having a higher activity (European patent application number 06112510.0) or one or more moderator layers (European patent application of Apr. 27, 2006 with the title “Verfahren zur Gasphasenoxidation unter Verwendung einer Moderatorlage” [Process for gas phase oxidation using a moderator layer] to BASF Aktiengesellschaft) may be used. The activity of the catalyst layers preferably increases continuously from the gas inlet side to the gas outlet side.

In the present invention, the activity of a catalyst layer is defined as follows: the higher the conversion for a specific reactant mixture at the same salt bath temperature, the higher the activity.

The bed length of the upper catalyst layer in a 3-layer catalyst system makes up preferably from 27 to 60%, in particular from 40 to 55%, of the total catalyst fuel height in the reactor. The bed length of the middle layer makes up preferably from 15 to 55%, preferably from 20 to 40%, of the total bed length.

In a 4-layer catalyst system, the upper layer makes up advantageously from 27 to 55%, in particular from 32 to 47%, the upper middle layer advantageously from 5 to 30%, preferably from 10 to 25%, and the lower middle layer advantageously from 8 to 35%, in particular from 12 to 30%, of the total bed height in the reactor. The lowermost layer of a 4-layer catalyst system makes up advantageously from 8 to 35%, in particular from 12 to 30%, of the total bed height in the reactor.

The catalyst layers may also, if appropriate, be distributed over several reactors. Typical reactors have a fuel height of from 2.5 to 3.4 meters.

The catalytically active composition of all catalysts preferably comprises at least vanadium oxide and titanium dioxide. Thus, the catalytically active composition may comprise oxidic compounds which, as promoters, influence the activity and selectivity of the catalyst, for example by lowering or increasing its activity.

Examples of activity-influencing promoters include the alkali metal oxides, especially cesium oxide, lithium oxide, potassium oxide and rubidium oxide, thallium(I) oxide, aluminum oxide, zirconium oxide, iron oxide, nickel oxide, cobalt oxide, manganese oxide, tin oxide, silver oxide, copper oxide, chromium oxide, molybdenum oxide, tungsten oxide, iridium oxide, tantalum oxide, niobium oxide, arsenic oxide, antimony oxide, cerium oxide. In general, from this group, cesium is used as the promoter. Useful sources of these elements include the oxides or hydroxides or the salts which can be converted thermally to oxides, such as carboxylates, especially the acetates, malonates or oxalates, carbonates, hydrogencarbonates or nitrates. Also suitable as activity-influencing promoters are oxidic phosphorus compounds, especially phosphorus pentoxide. Useful phosphorus sources include in particular phosphoric acid, phosphorous acid, hypophosphorous acid, ammonium phosphate or phosphoric esters and in particular ammonium dihydrogenphosphates. Suitable further activity-increasing additives are various antimony oxides, especially antimony trioxide.

Measures for controlling the activity of gas phase oxidation catalysts are known per se to the person skilled in the art. Advantageously, a higher activity of a catalyst layer is achieved by a lower content of cesium in the active composition, by a higher active composition per tube volume, by a lower content of vanadium in the active composition, by a higher BET surface area of the catalyst or by a combination of the means mentioned.

The catalysts used in the process according to the invention are generally coated catalysts in which the catalytically active composition is applied in coating form on an inert support. The layer thickness of the catalytically active composition is generally from 0.02 to 0.25 mm, preferably from 0.05 to 0.15 mm. In general, the catalysts have an active composition layer applied in coating form with essentially homogeneous chemical composition. In addition, it is also possible for two or more different active composition layers to be applied successively to a support. Reference is then made to a two-layer or multilayer catalyst (see, for example, DE 19839001 A1).

The inert support materials used may be virtually all prior art support materials, as are used advantageously in the preparation of coated catalysts for the oxidation of aromatic hydrocarbons to aldehydes, carboxylic acids and/or carboxylic anhydrides, as described, for example, in WO 2004/103561 on pages 5 and 6. Preference is given to using steatite in the form of spheres having a diameter of from 3 to 6 mm or of rings having an external diameter of from 5 to 9 mm, a length of from 4 to 7 mm and an internal diameter of from 3 to 7 mm.

The individual layers of the coated catalyst can be applied by any methods known per se, for example by spray application of solutions or suspensions in a coating drum, or coating with a solution or suspension in a fluidized bed, as described, for example, in WO 2005/030388, DE 4006935 A1, DE 19824532 A1, EP 0966324 B1.

The active composition of the upper catalyst layer(s) toward the gas inlet side, on nonporous and/or porous support material, contains from 7 to 11% by weights based on the overall catalyst, of active composition, comprising from 4 to 11% by weight of V₂O₅, from 0 to 4% by weight of Sb₂O₃ or Nb₂O₅, from 0 to 0.5% by weight of P, from 0.1 to 1.1% by weight of alkali (calculated as alkali metal), and, as the remainder, TiO₂ in anatase form.

The active composition of the middle catalyst layer(s) on nonporous and/or porous support material, contains from 7 to 11% by weight, based on the overall catalyst, of active composition, comprising from 5 to 13% by weight of V₂O₅, from 0 to 4% by weight of Sb₂O₃ or Nb₂O₅, from 0 to 0.5% by weight of P, from 0 to 0.4% by weight of alkali (calculated as alkali metal), and, as the remainder, TiO₂ in anatase form.

The active composition of the lower catalyst layer(s) toward the gas outlet side, on nonporous and/or porous support material, contains from 8 to 12% by weight, based on the overall catalyst, of active composition, comprising from 10 to 30% by weight of V₂O₅, from 0 to 4% by weight of Sb₂O₃ or Nb₂O₅, from 0 to 0.5% by weight of P, from 0 to 0.1% by weight of alkali (calculated as alkali metal), and, as the remainder, TiO₂ in anatase form.

The titanium dioxide used in anatase form advantageously has a BET surface area of from 5 to 50 m²/g, in particular from 15 to 40 m²/g. It is also possible to use mixtures of titanium dioxide in anatase form with different BET surface area, with the proviso that the resulting BET surface area has a value of from 15 to 40 m²/g. The individual catalyst layers may also comprise titanium dioxide with different BET surface areas. The BET surface area of the titanium dioxide used preferably increases from the upper catalyst layers toward the gas inlet to the lower catalyst layers toward the gas outlet.

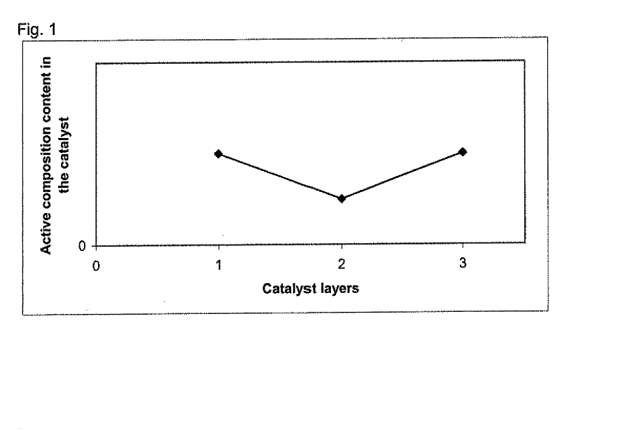

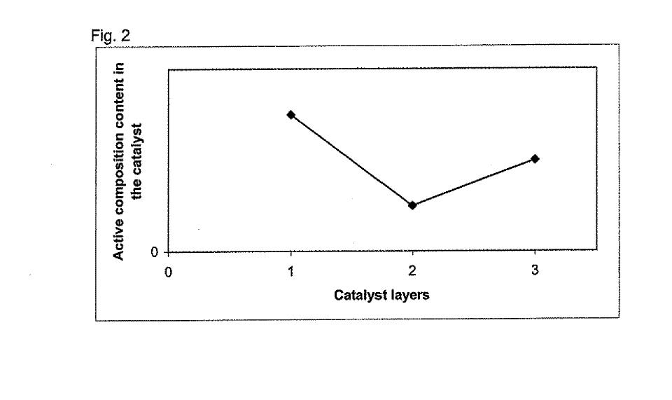

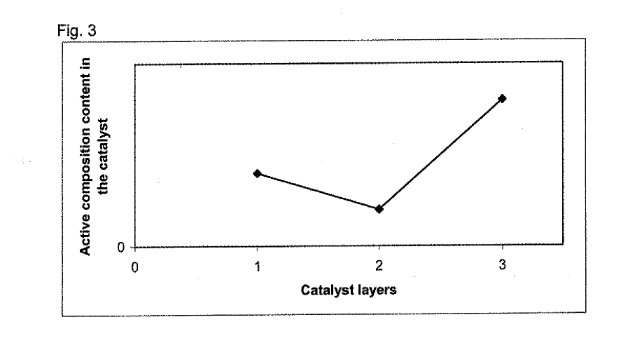

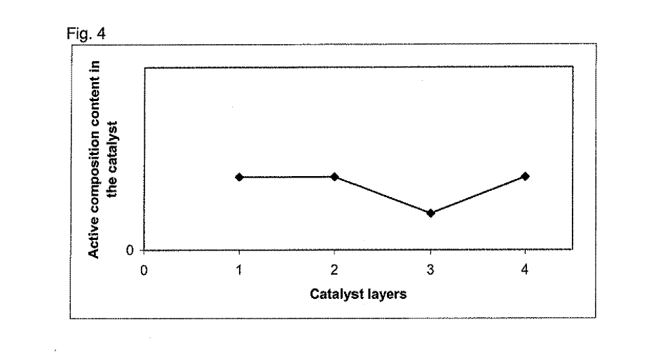

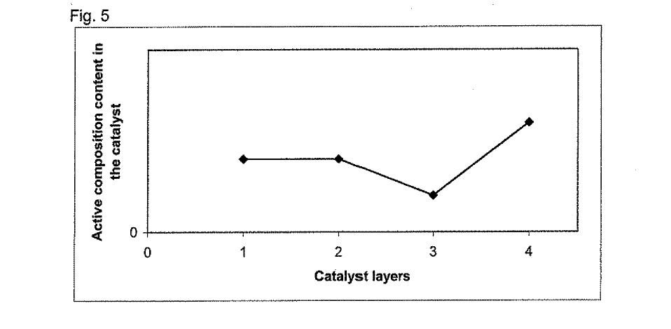

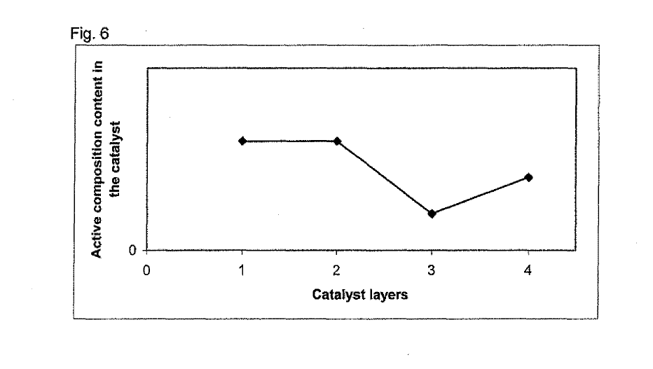

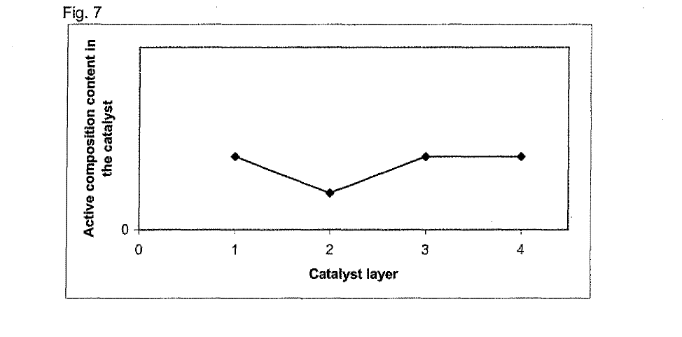

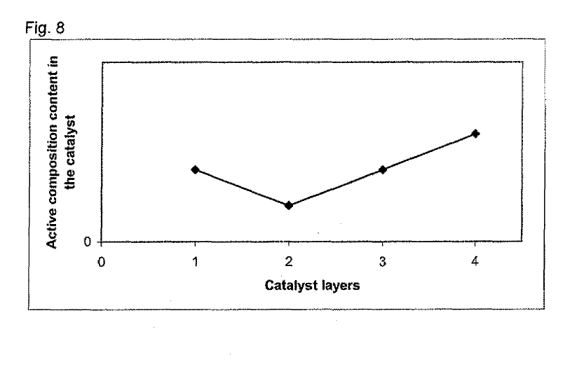

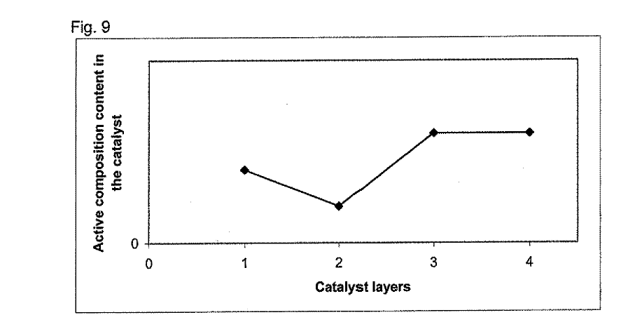

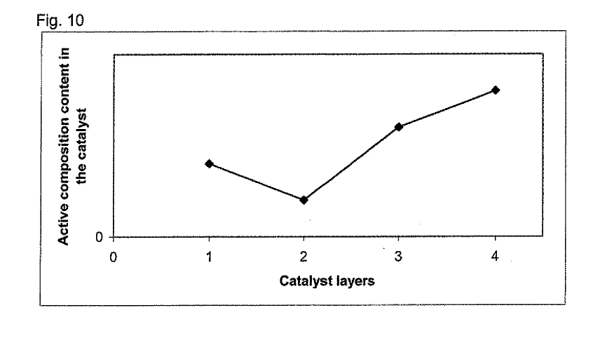

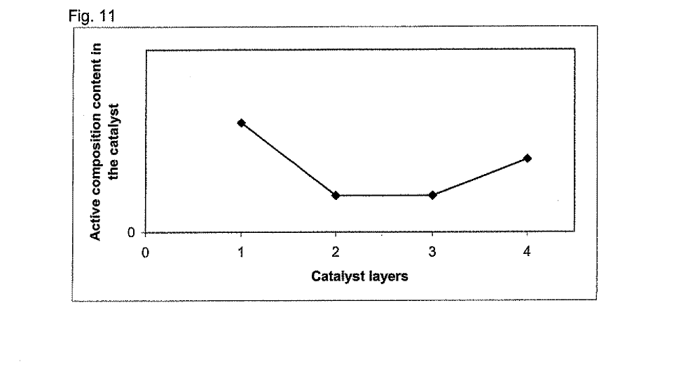

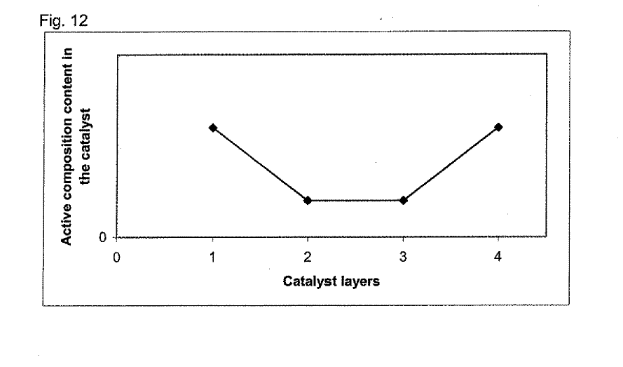

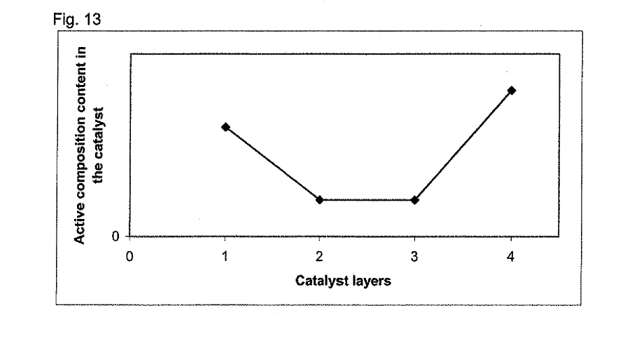

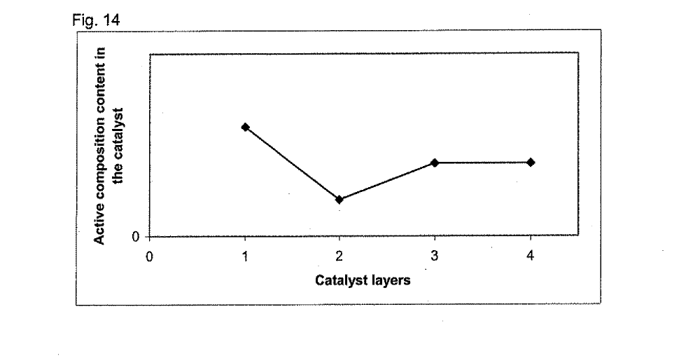

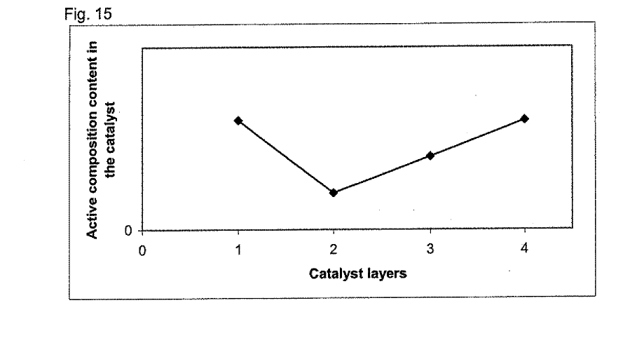

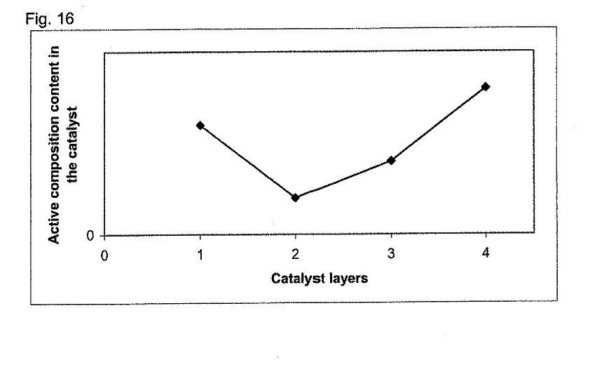

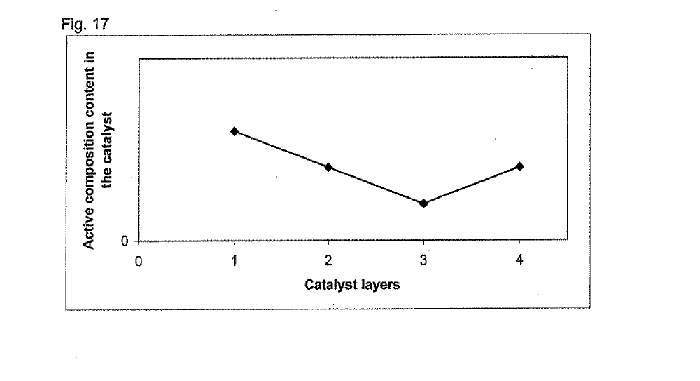

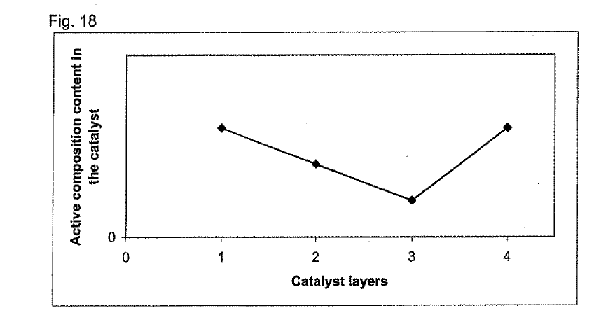

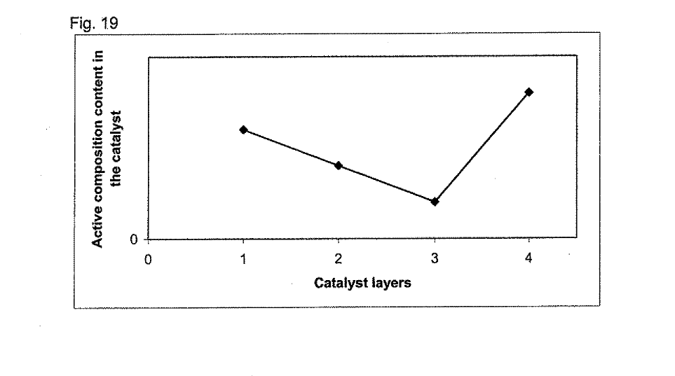

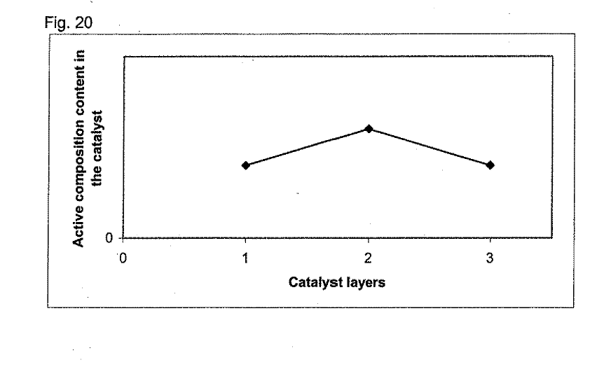

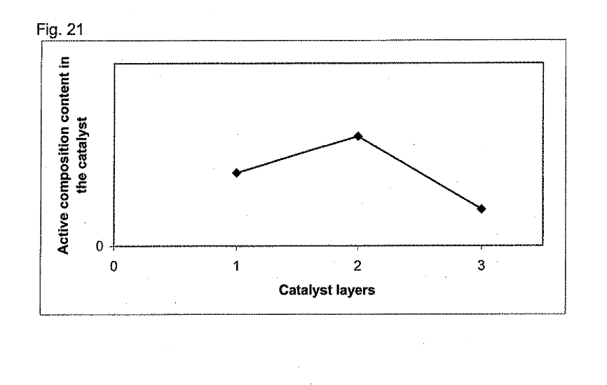

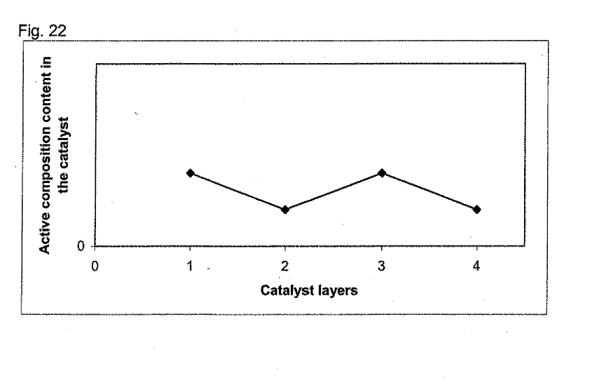

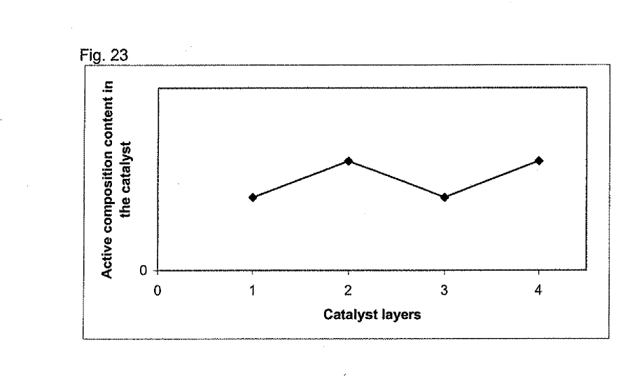

Inventive catalyst systems with three catalyst layers arranged one on top of the other in the reaction tube are, for example, shown in FIGS. 1 to 3. Also shown in FIGS. 4 to 19 are inventive catalyst systems with four catalyst layers arranged one on top of the other in the reaction tube. FIGS. 20 and 23 show noninventive catalyst systems which have to date not been described in the prior art.

For the reaction, the catalysts are charged layer by layer into the tubes of a tube bundle reactor. The catalysts of different activity can be thermostated to the same temperature or to different temperatures.

The present invention further relates to a process for gas phase oxidation, which comprises passing a gaseous stream which comprises a hydrocarbon and molecular oxygen through at least three catalyst layers arranged one on top of the other in the reaction tube, the active composition content, based on the total mass of the catalyst, of one or more of the middle catalyst layer(s) being lower than the active composition content of one or more of the upper catalyst layer(s) toward the gas inlet side and being lower than one or more of the lower catalyst layer(s) toward the gas outlet side.

The process according to the invention is suitable advantageously for the gas phase oxidation of aromatic C₆- to C₁₀-hydrocarbons, such as benzene, the xylenes, toluene, naphthalene or durene (1,2,4,5-tetramethylbenzene), to carboxylic acids and/or carboxylic anhydrides, such as maleic anhydride, phthalic anhydride, benzoic acid and/or pyromellitic anhydride.

In particular, the process is suitable for preparing phthalic anhydride from o-xylene and/or naphthalene. The gas phase reactions for preparing phthalic anhydride are common knowledge and are described, for example, in WO 2004/103561 on page 6.

### EXAMPLES

### 1. Preparation of the Catalysts

### Catalyst C1

After stirring for 18 hours, 417 g of a suspension consisting of 79.4 g of oxalic acid, 29.8 g of vanadium pentoxide, 7.64 g of antimony oxide, 2.36 g of cesium sulfate, 0.0 g of ammonium dihydrogenphosphate, 121.9 g of formamide, 380.9 g of titanium dioxide having a BET surface area of 20 m²/g and 578.7 g of water at 160° C. were applied together with 23.5 g of organic binder onto 1400 g of steatite rings of dimensions 8×6×5 mm (external diameter×height×internal diameter).

After calcination of the catalyst at 450° C. for one hour, the active composition applied to the steatite rings was 8.7%. The analyzed composition of the active composition consisted of 7.1% V₂O₅, 1.8% Sb₂O₃, 0.41% Cs, remainder TiO₂.

### Catalyst C2

Preparation analogous to C1 with variation of the composition of the suspension. After the catalyst had been calcined at 450° C. for one hour, the active composition applied to the steatite rings was 8.2%. The analyzed composition of the active composition consisted of 7.95% V₂O₅, 2.7% Sb₂O₃, 0.33% Cs, 0.1% P, remainder TiO₂.

### Catalyst C3

Preparation analogous to C1 with variation of the composition of the suspension. After the catalyst had been calcined at 450° C. for one hour, the active composition applied to the steatite rings was 8.2%. The analyzed composition of the active composition consisted of 7.1% V₂O₅, 2.4% Sb₂O₃, 0.14% Cs, 0.1% P, remainder TiO₂.

### Catalyst C4

Preparation analogous to C1 with variation of the composition of the suspension. After the catalyst had been calcined at 450° C. for one hour, the active composition applied to the steatite rings was 9.1%. The analyzed composition of the active composition consisted of 20% V₂O₅, 0.38% P, remainder TiO₂.

### Catalyst C5

Preparation analogous to C1 with variation of the composition of the suspension. After the catalyst had been calcined at 450° C. for one hour, the active composition applied to the steatite rings was 8.0%. The analyzed composition of the active composition consisted of 20% V₂O₅, 0.38% P, remainder TiO₂.

### 2. Axial Composition of the Catalyst System

### A) Inventive

The catalysts were introduced into a reactor tube with internal diameter 25 mm. Starting from the reactor inlet, the catalyst bed had the following composition: C1/C2/C3/C4=130/70/60/60 cm.

### B) Noninventive

The catalysts were introduced into a reactor tube with internal diameter 25 mm. Starting from the reactor inlet, the catalyst bed had the following composition: C1/C2/C3/C5=130/70/60/60 cm.

### 3. Catalytic Results

At the same volume flow rate (4 m³ (STP)/h), after running up to 80 g/m³ (STP), the following results were achieved:

| | Run | Salt bath | | | Residual |
|---|---|---|---|---|---|
| | time | temperature | PA yield | Phthalide | xylene |
| Catalyst | in days | in ° C. | in m/m % | % by wt. | % by wt. |
| A (inven- | 69 | 357 | 114.0 | 0.05 | 0.01 |
| tive) | | | | | |
| B (noninven- | 61 | 358 | 113.8 | 0.1 | 0.03 |
| tive) | | | | | |

1-7. (canceled)

8. A catalyst system for preparing one or more compounds selected from the group consisting of carboxylic acids, carboxylic anhydrides and mixtures thereof, the catalyst system comprising a reaction zone and a layered catalyst, wherein the reaction zone comprises a gas inlet region and a gas outlet region, wherein the layered catalyst comprises an active composition and comprises one or more middle layers, one or more first layers disposed on a side of the one or more middle layers toward the gas inlet region, and one or more second layers on a side of the one or more middle layers toward the gas outlet region, wherein the active composition content of one or more of the middle catalyst layers, based on total mass of the layered catalyst, is lower than the active composition content of the one or more first catalyst layers and is lower than one or more second catalyst layers.

9. The catalyst system according to claim 8, wherein the active composition content of the one or more middle layers is 0.1 to 5% by weight lower than that of the one or more first layers.

10. The catalyst system according to claim 8, wherein the active composition content of the one or more middle layers is 0.1 to 5% by weight lower than that of the one or more second layers.

11. The catalyst system according to claim 8, wherein the active composition content of the one or more middle layers is 0.1 to 5% by weight lower than that of the one or more first layers, and 0.1 to 5% by weight lower than that of the one or more second layers

12. The catalyst system according to claim 8, wherein the activity of the layered catalyst increases from the gas inlet region to the gas outlet region.

13. The catalyst system according to claim 11, wherein the activity of the layered catalyst increases from the gas inlet region to the gas outlet region.

14. The catalyst system according to claim 8,
wherein the one or more first layers comprise 7 to 11% by weight of the active composition, based on the layered catalyst, on a support material, the active composition comprising 4 to 11% by weight of V₂O₅, 0 to 4% by weight of Sb₂O₃ or Nb₂O₅, 0 to 0.5% by weight of P, 0.1 to 1.1% by weight of alkali (calculated as alkali metal), and TiO₂ in anatase form as the remainder;
wherein the one or more middle layers comprise 7 to 11% by weight of the active composition, based on the layered catalyst, on a support material, the active composition comprising 5 to 13% by weight of V₂O₅, 0 to 4% by weight of Sb₂O₃ or Nb₂O₅, 0 to 0.5% by weight of P, 0 to 0.4% by weight of alkali (calculated as alkali metal), and TiO₂ in anatase form as the remainder; and
wherein the one or more second layers comprise 8 to 12% by weight of the active composition, based on the layered catalyst, on a support material, the active composition comprising 10 to 30% by weight of V₂O₅, 0 to 4% by weight of Sb₂O₃ or Nb₂O₅, 0 to 0.5% by weight of P, 0 to 0.1% by weight of alkali (calculated as alkali metal), and TiO₂ in anatase form as the remainder.

15. The catalyst system according to claim 11,
wherein the one or more first layers comprise 7 to 11% by weight of the active composition, based on the layered catalyst, on a support material, the active composition comprising 4 to 11% by weight of V₂O₅, 0 to 4% by weight of Sb₂O₃ or Nb₂O₅, 0 to 0.5% by weight of P, 0.1 to 1.1% by weight of alkali (calculated as alkali metal), and TiO₂ in anatase form as the remainder;
wherein the one or more middle layers comprise 7 to 11% by weight of the active composition, based on the layered catalyst, on a support material, the active composition comprising 5 to 13% by weight of V₂O₅, 0 to 4% by weight of Sb₂O₃ or Nb₂O₅, 0 to 0.5% by weight of P, 0 to 0.4% by weight of alkali (calculated as alkali metal), and TiO₂ in anatase form as the remainder; and
wherein the one or more second layers comprise 8 to 12% by weight of the active composition, based on the layered catalyst, on a support material, the active composition comprising 10 to 30% by weight of V₂O₅, 0 to 4% by weight of Sb₂O₃ or Nb₂O₅, 0 to 0.5% by weight of P, 0 to 0.1% by weight of alkali (calculated as alkali metal), and TiO₂ in anatase form as the remainder.

16. The catalyst system according to claim 12,
wherein the one or more first layers comprise 7 to 11% by weight of the active composition, based on the layered catalyst, on a support material, the active composition comprising 4 to 11% by weight of V₂O₅, 0 to 4% by weight of Sb₂O₃ or Nb₂O₅, 0 to 0.5% by weight of P, 0.1 to 1.1% by weight of alkali (calculated as alkali metal), and TiO₂ in anatase form as the remainder;
wherein the one or more middle layers comprise 7 to 11% by weight of the active composition, based on the layered catalyst, on a support material, the active composition comprising 5 to 13% by weight of V₂O₅, 0 to 4% by weight of Sb₂O₃ or Nb₂O₅, 0 to 0.5% by weight of P, 0 to 0.4% by weight of alkali (calculated as alkali metal), and TiO₂ in anatase form as the remainder; and
wherein the one or more second layers comprise 8 to 12% by weight of the active composition, based on the layered catalyst, on a support material, the active composition comprising 10 to 30% by weight of V₂O₅, 0 to 4% by weight of Sb₂O₃ or Nb₂O₅, 0 to 0.5% by weight of P, 0 to 0.1% by weight of alkali (calculated as alkali metal), and TiO₂ in anatase form as the remainder.

17. The catalyst system according to claim 13,
wherein the one or more first layers comprise 7 to 1% by weight of the active composition, based on the layered catalyst, on a support material, the active composition comprising 4 to 11% by weight of V₂O₅, 0 to 4% by weight of Sb₂O₃ or Nb₂O₅, 0 to 0.5% by weight of P, 0.1 to 1.1% by weight of alkali (calculated as alkali metal), and TiO₂ in anatase form as the remainder;
wherein the one or more middle layers comprise 7 to 11% by weight of the active composition, based on the layered catalyst, on a support material, the active composition comprising 5 to 13% by weight of V₂O₅, 0 to 4% by weight of Sb₂O₃ or Nb₂O₅, 0 to 0.5% by weight of P, 0 to 0.4% by weight of alkali (calculated as alkali metal), and TiO₂ in anatase form as the remainder; and
wherein the one or more second layers comprise 8 to 12% by weight of the active composition, based on the layered catalyst, on a support material, the active composition comprising 10 to 30% by weight of V₂O₅, 0 to 4% by weight of Sb₂O₃ or Nb₂O₅, 0 to 0.5% by weight of P, 0 to 0.1% by weight of alkali (calculated as alkali metal), and TiO₂ in anatase form as the remainder.

18. A process for gas phase oxidation, comprising: providing a reaction zone having a gas inlet region and a gas outlet region and containing a layered catalyst comprising an active composition and having one or more middle layers, one or more first layers disposed on a side of the one or more middle layers toward the gas inlet region, and one or more second layers on a side of the one or more middle layers toward the gas outlet region; and passing a gaseous stream comprising a hydrocarbon and molecular oxygen through the layered catalyst; wherein the active composition content of one or more of the middle catalyst layers, based on total mass of the layered catalyst, is lower than the active composition content of the one or more first catalyst layers and is lower than one or more second catalyst layers.

19. The process according to claim 18, wherein the active composition content of the one or more middle layers is 0.1 to 5% by weight lower than that of the one or more first layers.

20. The process according to claim 18, wherein the active composition content of the one or more middle layers is 0.1 to 5% by weight lower than that of the one or more second layers.

21. The process according to claim 18, wherein the active composition content of the one or more middle layers is 0.1 to 5% by weight lower than that of the one or more first layers, and 0.1 to 5% by weight lower than that of the one or more second layers

22. The process according to claim 18, wherein the activity of the layered catalyst increases from the gas inlet region to the gas outlet region.

23. The process according to claim 21, wherein the activity of the layered catalyst increases from the gas inlet region to the gas outlet region.

24. The process according to claim 18,
wherein the one or more first layers comprise 7 to 11% by weight of the active composition, based on the layered catalyst, on a support material, the active composition comprising 4 to 11% by weight of V₂O₅, 0 to 4% by weight of Sb₂O₃ or Nb₂O₅, 0 to 0.5% by weight of P, 0.1 to 1.1% by weight of alkali (calculated as alkali metal), and TiO₂ in anatase form as the remainder;
wherein the one or more middle layers comprise 7 to 11% by weight of the active composition, based on the layered catalyst, on a support material, the active composition comprising 5 to 13% by weight of V₂O₅, 0 to 4% by weight of Sb₂O₃ or Nb₂O₅, 0 to 0.5% by weight of P, 0 to 0.4% by weight of alkali (calculated as alkali metal), and TiO₂ in anatase form as the remainder; and
wherein the one or more second layers comprise 8 to 12% by weight of the active composition, based on the layered catalyst, on a support material, the active composition comprising 10 to 30% by weight of V₂O₅, 0 to 4% by weight of Sb₂O₃ or Nb₂O₅, 0 to 0.5% by weight of P, 0 to 0.1% by weight of alkali (calculated as alkali metal), and TiO₂ in anatase form as the remainder.

25. The process according to claim 18, wherein the hydrocarbon comprises one or more compounds selected from the group consisting of xylene, naphthalene and mixtures thereof.
